(19) United States
(12) Patent Application Publication
(10) Pub. No.: US 2011/0172419 A1
(43) Pub. Date: Jul. 14, 2011

(54) PROCESS FOR THE SYNTHESIS OF CYCLIC CARBAMATES

(76) Inventors: Aleksander Warm, Arbaz (CH); Miriam Lorenzi, Naters (CH); Gareth Griffiths, Visp (CH)

(21) Appl. No.: 12/741,067

(22) PCT Filed: Apr. 9, 2010
(86) PCT No.: PCT/EP2010/002226

§ 371 Date: Mar. 4, 2011

Related U.S. Application Data
(60) Provisional application No. 61/167,897, filed on Apr. 9, 2009.

(30) Foreign Application Priority Data
Apr. 9, 2009 (EP) 09005224.2

(51) Int. Cl. C07D 263/58

(52) U.S. Cl. 544/97

(57) ABSTRACT

The invention is directed to a process for the preparation of cyclic carbamates of formula

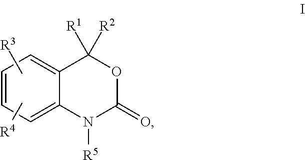

and/or a salt thereof,
from the corresponding o-aminobenzyl alcohol and/or a salt thereof.

The invention is directed to a process for the preparation of compounds of formula

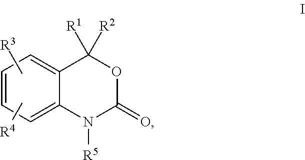

and salts thereof, wherein R¹ through R⁵ are as defined below.

Some of the cyclic carbamates of formula I are key intermediates for the preparation of pharmaceuticals and agrochemicals.

WO-A-98/27073 provides a cyclisation reaction of an o-aminobenzyl alcohol of the formula

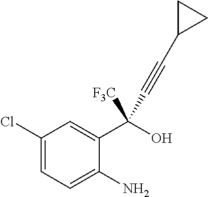

with phosgene in an organic solvent system containing heptanes and tetrahydrofuran. WO-A-98/51676 and WO-A-99/61026 provide a related cyclisation process of such an o-aminobenzyl alcohol with phosgene in a biphasic solvent system comprising methyl tent-butyl ether/water or toluene/water in the presence of potassium hydrogencarbonate.

The problem to be solved was to supply an alternative process for the production of the compound of formula I in high yield and quality.

The problem is solved by the process of claim 1.

Provided is a process for the preparation of a compound of formula

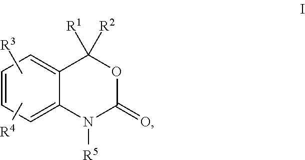

and/or a salt thereof,
wherein R¹ and R² are independently selected from the group consisting of hydrogen, C₁₋₆-alkyl, (C₁₋₆-alkoxy)carbonyl, C₂₋₆-alkenyl, C₂₋₆-alkynyl and C₃₋₆-cycloalkyl, wherein each alkyl, alkoxy, alkenyl, alkynyl and cycloalkyl can carry a further substituent selected from the group consisting of aryl, aralkyl, C₁₋₆-alkyl and (1′-R⁶)-C₃₋₆-cycloalkyl, wherein R⁶ is hydrogen, methyl or ethyl, and wherein each such further substituent is optionally substituted with one or more halogen atoms, with the proviso that at least one of the residues R¹ and R² is different from hydrogen,
R³ and R⁴ are independently selected from the group consisting of hydrogen, halogen, and

C₁₋₆-alkyl, optionally the latter being substituted with one or more halogen atoms, and R⁵ is hydrogen or a substituent selected from the group consisting of aryl, aralkyl, C₁₋₆-alkyl and (C₁₋₆-alkoxy)carbonyl, wherein the aryl moiety in any aryl or aralkyl group is optionally substituted with one or more C₁₋₆-alkyl, C₁₋₆-alkoxy or C₃₋₈-cycloalkyl groups, each said alkyl, alkoxy or cycloalkyl group optionally being substituted with one or more halogen atoms,

said process comprising the reaction of
a compound of formula

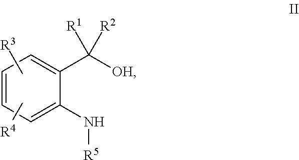

and/or a salt thereof,
wherein R¹, R², R³, R⁴, and R⁵ are as defined above,
with a phosgene equivalent selected from the group consisting of phosgene, diphosgene or triphosgene, or a mixture thereof;
characterized in that the reaction is carried out in the presence of water and at least one water-miscible organic solvent selected from the group consisting of tetrahydrofuran, dioxane, acetonitrile, C₁₋₄-alcohols, dimethoxyethane, diethoxyethane and dimethyl sulfoxide, wherein the pH is in the range of 6 to 11.

Herein the term “alkyl” represents a linear or branched alkyl group. By using the form “C₁₋ₙ-alkyl” is meant the main chain of the alkyl group having 1 to n carbon atoms. C₁₋₆-alkyl represents for example methyl, ethyl, propyl, isopropyl, butyl, isobutyl, sec-butyl, tent-butyl, pentyl or hexyl.

Herein the term “alkenyl” represents a linear or branched group carrying at least one carbon-carbon double bound. By using the form “C₂₋ₙ-alkenyl” is meant the main chain of the alkenyl group having 2 to n carbon atoms. C₂₋₆-alkenyl represents for example ethenyl (vinyl), propen-2-yl, propen-3-yl (allyl), buten-1-yl or hexen-1-yl.

Herein the term “alkynyl” represents a linear or branched group carrying at least one carbon-carbon triple bound. By using the form “C₂₋ₙ-alkynyl” is meant the main chain of the alkynyl group having 2 to n carbon atoms. C₂₋₆-alkynyl represents for example ethinyl, 1-propynyl, 3-propynyl or 1-hexynyl.

Herein the term “alkoxy” represents a linear or branched alkoxy group. By using the form “C₁₋ₙ-alkoxy” the alkyl group is meant having 1 to n carbon atoms. C₁₋₆-alkoxy represents for example methoxy, ethoxy, propoxy, isopropoxy, butoxy, isobutoxy, sec-butoxy, tent-butoxy, pentyloxy and hexyloxy.

Herein the term “C₃₋ₙ-cycloalkyl” represents a cycloaliphatic group having 3 to n ring carbon atoms. C₃₋₈-cycloalkyl is selected from the group consisting of cyclopropyl, cyclobutyl, cyclopentyl, cyclohexyl, cycloheptyl and cyclooctyl.

Herein the term “aryl” represents an aromatic or heteroaromatic group, selected from the group consisting of phenyl, naphth-1-yl, naphth-2-yl, furan-2-yl, furan-3-yl, thiophen-2-yl, thiophen-3-yl, benzo[b]furan-2-yl and benzo[b]thiophen-2-yl.

Herein the term “aralkyl” represents a group consisting of an alkyl and an aryl moiety, wherein the alkyl moiety of the aralkyl residue is a C₁₋₈alkyl group and the aryl moiety is selected from the group consisting of phenyl, 1-naphthyl, 2-naphthyl, furan-2-yl, furan-3-yl, thiophen-2-yl, thiophen-3-yl, benzo[b]furan-2-yl and benzo[b]thiophen-2-yl.

Beside phosgene (Carbonyl chloride, COCl₂, CAS No. 75-44-5) there exists two related “dimeric” and “trimeric” compounds, i.e. diphosgene (Trichloromethyl chloroformate, C₂Cl₄O₂, CAS No. 503-38-8) and triphosgene (Bis(trichloromethyl) carbonate, C₃Cl₆O₃, CAS No. 32315-10-9). It is well known that the latter two, from a chemist's point of view, can be regarded as phosgene equivalents, which are more conveniently to handle but possesses the same reactivity. Phosgene, diphosgene or triphosgene are gaseous, liquid or solid under standard conditions (20° C., 1 bar), respectively. Each compound can be used in chemical reactions neat or dissolved in a suitable solvent. They also can be used as a mixture of two or three. One mol of triphosgene has the same effect then three moles of phosgene, while diphosgene has the same effect then two moles of phosgene. Thus, necessary molar amounts of a mixture can be calculated easily. Diphosgene and triphosgene have the advantage of easier dosing and handling in an undeveloped industrial area. Nevertheless, since diphosgen and triphosgene easily develop phosgene for example even in the presence of humid air, the security measurements have to be on the same high level to protect humans, animals and environment. The workup procedures for removal of excess phosgene (and phosgene equivalents if still present) and organic solvents to facilitate crystallization are preferably carried out as known in the art.

Above pH 11 and below pH 6 increased formation of by-products occurs. Adjustment of the pH can be carried out for example by pre-charging a suitable base in the reaction vessel and/or by controlled addition of a suitable base, preferably by addition of an aqueous sodium and/or a potassium hydroxide solution.

Where more than one organic solvent is present, the at least one water-miscible organic solvent has to act as solubilizer providing control of the pH in the liquid phase. Preferably the mixture is a homogeneous aqueous solution or suspension under standard conditions (20° C., 1 bar). Towards complete conversion of the starting material—i.e. near at the end of the reaction—it is possible that the pH may drop below pH 6 due to an excess of phosgene. Therefore, pH control in the range of pH 6 to 11 should be provided until at least 90% conversion. The conversion can be determined quickly by standard methods. Short time excursion of the prescribed pH range during the reaction is possible without being outside the scope of the invention.

When a chiral o-aminobenzyl alcohol is used as a starting compound in the process, i.e. in compounds where R¹ and R² are not identical, the confirmation of the starting compound is maintained in the compound of formula I. In a preferred embodiment the reaction is carried out with compounds where R¹ and R² are not identical.

In a further preferred embodiment in compound of formula II the substituent R¹ is C₁₋₄-perfluoroalkyl, R² is 2-cyclopropyl-ethynyl or 2-(1-methyl-cyclopropyl)-ethynyl, R³ is a halogen atom in para-position to the amino group, preferably chlorine, and R⁴ is hydrogen.

In a another preferred embodiment in compound of formula II the substituent R¹ is C₁₋₄-perfluoroalkyl, R² is 2-cyclopropyl-ethynyl or 2-(1-methyl-cyclopropyl)-ethynyl, R³ is a halogen in para-position to the amino group, preferably chlorine, and R⁴ and R⁵ are hydrogen.

The reaction can be carried out with the free base of formula II as starting compound or a salt of said base with an inorganic or organic acid. Suitable salts are for example hydro-chlorides, sulfonates, methanesulfonates, oxalates or tartrates. Also useful are non stoichiometric mixtures of the compound of formula II and at least one acid. Usually such mixtures contain excess amounts of acid. A preferred salt is a methanesulfonate, more preferably a mixture containing 1.5 molar equivalents of methanesulfonic acid.

The phosgene equivalents phosgene, diphosgene and triphosgene may be provided in gasous, liquid or solid form or dissolved in an organic solvent. In a preferred embodiment it is provided in gaseous form. In another preferred embodiment it is provided in liquid form. In yet another preferred embodiment it is provided in solid form.

In order to improve workup procedure it might be useful to supply phosgene (phosgene equivalent) in slight excess. Preferably the molar ratio of the phosgene equivalent, calculated as monomeric phosgene amount, to the compound of formula II is in a range of 1:1 to 2.5:1, more preferably in the range of 1.1:1 to 1.5:1. Generally, the most preferred molar ratio is about 1.2:1 calculated as phosgene.

The base used in the reaction can be an inorganic or organic base. Examples for inorganic bases are alkali or earth alkali metal carbonates, hydrogen carbonates and hydroxides.

Examples of suitable organic bases are piperidine, C₁₋₄-alkylpiperidines, pyridine, C₁₋₄-alkylpyridines, morpholine or tri-C₁₋₄-alkylamines, wherein any of the alkyl moieties are independently selected from the group consisting of methyl, ethyl, propyl, isopropyl, butyl, isobutyl, sec-butyl and tent-butyl. Using weak bases like alkali or earth alkali metal carbonates, hydrogen carbonates or a combination of different bases with different pK_{b} establishes a buffered system wherein the pH can be easily controlled. Using strong bases like alkali or earth alkali metal hydroxides may require parallel dosage of phosgene and the base to maintain the pH in the prescribed range.

In a preferred embodiment the weight ratio of water to the water-miscible organic solvent(s) is in the range from 1.5:1 to 5:1, preferably in the range from 2:1 to 3.5:1.

The most suitable organic solvent comprises tetrahydrofuran and mixtures thereof.

Herein the term “C₁₋₄-alcohol” represents an alcohol selected from the group consisting of methanol, ethanol, propanol, isopropyl alcohol, butanol, isobanol, sec-butanol, tert-butanol, Cl₃CCH₂OH, CF₃CH₂OH, CH₂═CHCH₂OH or fully alkylated amino C₁₋₄-alcohols such as (CH₃)₂NCH₂CH₂OH.

In a preferred embodiment the reaction is carried out at a temperature from −30 to +40° C. until completion of the reaction, preferably in the range from −30 to +30° C.

### EXAMPLES

If not otherwise mentioned in examples 1 to 11, during the phosgene addition the pH is adjusted within the range of 6 to 11 by addition of an aqueous solution of sodium hydroxide. Because phosgene is readily available for the applicant experiments with other phosgene equivalents then phosgene itself have not been carried out because of the well known equivalence of all three available forms. Adjustment of the pH is carried out by addition of the base pre-charged in the reaction. Heptanes in the meaning of the present invention and the experiments means any mixture of linear and branched heptanes, comprising n-heptane as the major component of at least 50%, preferably of at least 70%, more preferably of at least 90% and even more preferably of at least 95%.

### Example 1

An aromatic amino alcohol of formula II (R¹=R²R³=R⁴=R⁵=hydrogen, 24.11 g, 196 mmol) was dissolved in THF (151 g) and charged with potassium hydrogen carbonate (60.8 g, 607 mmol) and water (293 g). The agitated brown mixture was cooled to about 12° C. and gaseous phosgene (23.3 g, 236 mmol) was added within 1 h wherein the temperature in the reaction vessel was kept at 7 to 17° C. At the end of the phosgene addition the reaction mixture was additionally agitated for 1 h at 12° C. A conversion of 95.0% was reached. Then the aqueous phase was separated at 12° C. The organic phase was concentrated to dryness (20 to 45° C., 20 to 90 mbar). Heptanes (512 g) were added to the light brown fluffy solid residue and the temperature increased to 69° C. The slurry was cooled to −10° C. in 1 hour and stirred at this temperature for 30 min. The filter cake was washed with cold heptane. After drying 84.7% of compound of formula I (R¹=R²=R³=R⁴=R⁵=hydrogen, 24.7 g) has been obtained.

### Example 2

An aromatic amino alcohol of formula II (R¹=phenyl, R²=R³=R⁴=R⁵=hydrogen, 2.0 g, 10 mmol) was dissolved in dimethoxyethane (4.3 g) as the water miscible solvent and slowly charged with an solution of potassium carbonate (3.2 g, 23 mmol) in water (15 g). The agitated yellow mixture was cooled to about 20° C. and gaseous phosgene (2.3 g, 23 mmol) was added within 20 min wherein the temperature in the reaction vessel was kept at 20 to 30° C. At the end of the phosgene addition the reaction mixture was agitated for additional 30 min at 20° C. The mixture was heated to about 30 to 35° C. and the aqueous phase was separated. The organic phase was concentrated to dryness (40 to 45° C., at <40 mbar). After drying, 88.0% of compound of formula I (R¹=phenyl, R²=R³=R⁴=R⁵=hydrogen, 1.99 g) was obtained.

### Example 3

Example 2 was repeated starting with a compound of formula II (R¹=R²=trifluoro-methyl, R³=R⁴=R⁵=hydrogen, 2.1 g, 8 mmol) using sodium carbonate as base (2 g, 19 mmol), the amount of phosgene (0.95 g, 9.6 mmol) and THF (4.4 g) as the water miscible solvent. After drying, 80.1% of compound of formula I (R¹=R²=trifluoro-methyl, R³=R⁴=R⁵=hydrogen, 1.85 g) was obtained.

### Example 4

An aromatic amino alcohol of formula II (R¹=R²=R³=R⁴=R⁵=hydrogen, R³=3-methyl, 13.48 g, 98 mmol) was dissolved in a THF/diethoxymethane mixture (1:1, v:v) (73 g) and charged with water (93 g). The agitated brown mixture was cooled to about 12° C. A parallel dosage of gaseous phosgene (13.7 g, 139 mmol) and 25% aqueous NaOH (62.9 g, 393 mmol) was performed within 1 h wherein the pH was kept between 8 and 9 and the temperature in the reaction vessel was kept at 7 to 17° C. At the end of the phosgene addition the reaction mixture was additionally agitated for 1 h at 12° C. After workup procedure according to example 1 and drying 100% of compound of formula I (R¹=R²=R³=R⁴=R⁵=hydrogen, 16.19 g) was obtained.

1. A process for the preparation of a compound of formula 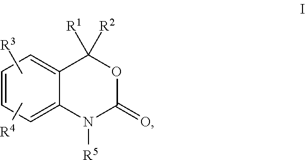
and/or a salt thereof,
wherein R¹ and R² are independently selected from the group consisting of hydrogen, C₁₋₆-alkyl or (C₁₋₆-alkoxy) carbonyl, any of said alkyl or alkoxy optionally being substituted with one or more halogen atoms, R² is selected from the group consisting of hydrogen, C₁₋₆-alkyl, (C₁₋₆-alkoxy) carbonyl, C₂₋₆-alkenyl, C₂₋₆-alkynyl and C₃₋₆-cyclo-alkyl, wherein any of said alkyl, alkoxy, alkenyl, alkynyl and cycloalkyl can carry a further substituent selected from the group consisting of aryl, aralkyl, C₁₋₆-alkyl and (1′-R⁶)-C₃₋₆-cycloalkyl, wherein R⁶ is hydrogen, methyl or ethyl, and wherein any of such further substituent is optionally substituted with one or more halogen atoms, R³ and R⁴ are independently selected from the group consisting of hydrogen, halogen atom, and C₁₋₆-alkyl, optionally substituted with one or more halogen atoms, and R⁵ is hydrogen or a group selected from the group consisting of aryl, aralkyl, C₁₋₆-alkyl and (C₁₋₆-alkoxy)carbonyl, wherein the aryl moiety in any aryl or aralkyl is optionally substituted with one or more C₁₋₆-alkyl, C₁₋₆-alkoxy or C₃₋₈-cycloalkyl, each alkyl, alkoxy or cycloalkyl substituent optionally being substituted with one or more halogen atoms,
said process comprising the reaction of
a compound of formula 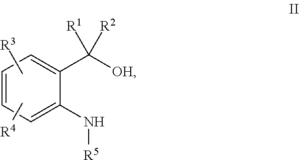
and/or a salt thereof,
wherein R¹, R², R³, R⁴ and R⁵ are as defined above,
with reacted with a phosgene equivalent selected from the group consisting of phosgene, diphosgene or triphosgene, or a mixture thereof;
characterized in that the reaction is carried in the presence of a base, water and at least one water miscible organic solvent selected from the group consisting of tetrahydrofuran, dioxane, acetonitrile, C₁₋₄-alcohols, dimethoxyethane, diethoxyethane and dimethyl sulfoxide, and wherein the pH is in the range of 6 to 11.

2. The process of claim 1, wherein the phosgene equivalent is provided in gaseous form.

3. The process of claim 1, wherein the phosgene equivalent is provided in liquid form.

4. The process of claim 1, wherein the phosgene equivalent is provided in solid form.

5. The process of claim 1, wherein the molar ratio of the phosgene equivalent, calculated as monomeric phosgene amount, to the compound of formula II is in a range of 1:1 to 2.5:1.

6. The process of claim 1, wherein the base is an inorganic or organic base selected from the group consisting of alkali or earth alkali metal carbonates, hydrogen carbonates and hydroxides, piperidine, C₁₋₄-alkylpiperidines, pyridine, C₁₋₄-alkylpyridines, morpholine and tri-C₁₋₄-alkylamines.

7. The process of claim 1, wherein the weight ratio of water to the organic solvent(s) is in the range from 1.5:1 to 5:1.

8. The process of claim 1, wherein the organic solvent is tetrahydrofuran.

9. The process of claim 1, wherein the reaction is carried out at a temperature from −30 to +40° C.
